(19) United States
(12) Patent Application Publication
(10) Pub. No.: US 2022/0134106 A1
(43) Pub. Date: May 5, 2022

(54) BODY REGION DEPENDENT ELECTRIC STIMULATION BASED-DEVICE FOR MEASURING BIOLOGICAL SIGNALS

(71) Applicant: Lee Sol Co., Ltd., Seoul (KR)

(72) Inventors: Moonsoo KIM, Gyeonggi-do (KR); Seung Woo LEE, Seoul (KR)

(21) Appl. No.: 17/405,394

(22) Filed: Aug. 18, 2021

(30) Foreign Application Priority Data
Nov. 2, 2020 (KR) 10-2020-0144508
Jun. 22, 2021 (KR) 10-2021-0081019

(51) Int. Cl. A61N 1/36; A61N 1/04; A61N 1/20

(52) U.S. Cl. CPC A61N 1/36031; A61N 1/0456; A61N 1/0476; A61N 1/20; A61N 1/36025; A61N 1/36034; A61N 1/0484

(57) ABSTRACT

The present invention relates to an electric current stimulation device capable of selectively stimulating a region including: a main body; an electrode unit which is equipped to the main body to contact at least a part of a user's body; and a control unit which controls the electrode unit to transfer a microcurrent of 1 mA or less by a Cranial Electrotherapy Stimulation (CES) method, wherein the control unit determines at least one stimulation area required to be stimulated in whole area of the user's brain according to a type of the user's diseases and determines at least one of two or more of first electrodes required to be activated (ON) in a plurality of electrodes, an intensity of the microcurrent which respective electrodes of the first electrode should induce and a route thereof in order to treat the disease focusing the microcurrents on the stimulation area. Accordingly, the present invention allows intensively treating a more accurate area in an appropriate level and providing a more efficient treatment environment to the stimulation area.

### CROSS-REFERENCE TO RELATED APPLICATIONS

The present application claims priority to Korean Patent Application Nos. 10-2020-0144508, filed on Nov. 2, 2020, and 10-2021-0081019, filed on Jun. 22, 2021, the entire contents of which are incorporated herein by reference.

### BACKGROUND

### Technical Field

The present invention relates to a body region dependent electric stimulation-based device for measuring biological signals, particularly to a body region dependent electric stimulation-based device for measuring biological signals, which measures a change in biological signals for a part of a user's body to generate an optimum electric stimulation signal, followed by transferring the optimum electric stimulation signal to a part of the user's body, thereby maximizing a treatment effect thereon.

Further, the present invention relates to an electric stimulation device capable of selectively stimulating a region and a controlling method thereof. The device is capable of intensively treating a more precise region with an appropriate intensity of an electric current through a configuration that selects a treatment target region requiring stimulation depending on the type of the user's disease, optionally activates a plurality of electrodes for focusing microcurrents to the selected treatment target region and optionally determines an intensity of the microcurrent which the activated electrode should induce and a route thereof thereby providing the treatment target region with an environment for efficient treatment.

### Description of the Related Art

Generally, the microcurrent refers to an electric current below 1000 μA and a bioelectric current corresponding to 40-60 μA flows in a human body for transferring signals between each organ inside the body. Commonly, when the body is in an unstable state, the bioelectric currents flowing in the human body become weakened. In order to compensate the weakened bioelectric currents, various types of treatment methods have been developed which allow electric currents to forcibly flow to the human body from the exterior to help recovering a physical capability. For example, it has been reported that the microcurrent has effects such as an adenosine triphosphate generation increment, fracture union facilitation, blood circulation improvement, a diabetes treatment, an osteoporosis treatment and an arthritis treatment.

As forcibly flowing the microcurrent to the human body, the effects resulting therefrom may be classified into blood flow increment in the capillary and microcirculation increment in the human body. The effect resulting from the blood flow increment in the capillary includes, for example, blood circulation improvement, muscle fatigue recovery, pain alleviation, amelioration of peripheral nerve, facilitation of growth and development, facilitation of cell activity, facilitation of osteogenesis, etc. The effect resulting from the microcirculation increment in the human body includes, for example, adipocyte lysis, collagen synthesis increase, metabolism enhancement, activation of neuroendocrine hormone, amelioration of edema, etc. Further, reported were clinical cases due to stimulation by the microcurrent forcibly flowing in the human body. These cases include a microcurrent treatment which controls pains, inflammation and edema and adipocyte lysis which disrupts adipocytes by flowing the microcurrent to an acupoint of a mast cell. Particularly, reported was that electric stimulation in the fibroblast results in increasing production of collagens and propagating fibroblasts.

Another effect resulting from forcibly flowing the microcurrent is to prevent alopecia. It is reported that when flowing the microcurrent to a scalp, scalp cells are stimulated to stimulate follicular cells, consequently allowing blood circulation in the scalp and this resulting in nourishing the scalp sufficiently, allowing preventing alopecia.

As a prior art for inducing the microcurrent flow in the human body, provided is Korean Patent Application Publication No. 2005-0030450 “Ornaments with health promotion by microcurrent”. This prior art relates to a neural system which flows microcurrents of 200-300 μA to the human body through various accessories (or ornaments) such as necklaces, rings or timepieces which directly contact to the human body. The ornaments with health promotion by microcurrent according to the prior art is provided, which installs a solar cell is mounted in an ornament part of a necklace to obtain the microcurrents; which allows the output of the solar cell approximating 500-800 μA with respect to the time at which a solar intensity is the highest to charge or discharge a recharge portion; which connects a chipped constant current circuit that is coupled to the recharge portion and makes currents of the recharge portion constant to 200-300 μA equivalent to bioelectric currents; which flows one pole of the microcurrents being constant to 200-300 μA to the vicinity of the chest of the human body through a connection terminal that contacts to the human body; and in which an insulation material is filled between the connection terminal and the ornament portion.

As another prior art for inducing the microcurrent flow, provided is Korean Patent No. 0795830 “Functional shoes”. This prior art relates to functional shoes comprising: an insole with at least one penetrating insert holes; an acupressure member that is inserted into the insert hole to apply acupressure to the sole of user's foot; a spring that is positioned in a lower part of the acupressure member to support the acupressure; a printed circuit film which is attached to the lower part of the insole and which an interconnect circuit electrically connected with the spring is printed on; a hard case that accommodates and protects the printed circuit film and attached to the insole to shape the sole of the user's foot; and one or more electric generators which are positioned in an outsole of the shoe to provide electric stimulation to the sole of the user's foot.

As another prior art for inducing the microcurrent flow, provided is Korean Patent No. 0938605 “Clothing built-in with stimulus device of minuteness electric”. This prior art relates to a clothing built-in with stimulus device of minuteness electric comprising: a rectangular shaped main body in which a space is formed; a disc shaped fluid magnet that flows into the space; a coil that is installed in a lateral surface of the main body and generates microcurrents and a magnetic field by interaction with the fluid magnet; a cover that is installed at both ends of the main body and prevents leakage of the fluid magnet; and a microcurrent stimulation device that is composed of a stimulation member which is installed at both ends of the coil and applies microcurrent stimulation to the human body, in which a main body of the microcurrent stimulation device is installed in one side of a sleeve of the clothing and the stimulation member is installed exposed to the surface contacting with an acupoint of a wrist of and a skin of the human body.

However, the aforementioned prior arts fail to reflect changes in the user's biological signals and thus they are not available to generate an optimum electric signal for maximizing treatment effects on a part of the user's body.

Recently, brain related diseases, such as sleep disorder, dementia, depression, etc., have been increased. It is very difficult to treat such diseases and these result in drug overdoses. What is worse, caused are side effects (e.g., drug addiction, sleep-acting, dysuria, etc.) due to sleeping pills, antidepressants, etc.

That is, it is expected that those brain related diseases, such as sleep disorder, dementia, depression, etc., will be increased. Thus, required is a paradigm change for the brain science in order to replace existing treatments such as hormone based chemical therapy.

Generally, microglia are a type of neuroglia located throughout the brain, performing the function of leukocytes in the brain. Appropriate activities of the microglia can maintain brain heath.

On the other hand, over increased activities of the microglia may cause depression increases, hypomnesis, decline cognitive functions, etc. Accordingly, it is a key for treating various types of brain related diseases to control the activity of the microglia to an appropriate level.

Required is the most advanced therapy which has high compatibility with the existing chemical therapy facilitating or suppressing hormone releases, is capable of controlling the microglia safer and more effectively and allows mid- to long-term treatment without side effects.

For this, as a method for controlling the activity to an appropriate level, provided is a new paradigm such as Transcranial Magnetic Stimulation (TMS), neurofeedback, gamma ray therapy, improved concussion protocol, Fasting Mimicking Diet (FMD), etc.

For example, Deep-Brain Stimulation (DBS) that applies electric stimulation to a correct region is an invasive stimulation method inserting stimulation physically. Thus, this accompanies a surgery. Further, non-invasive stimulation methods such as a TMS method using a magnet, a US method using an ultrasonic wave, a TDCS method using an electrode are not available to apply stimulation to a target region accurately.

In addition, in a case of a HD-TDCS method upgrading the TDCS method, there are limitations such as moving an electrode along a desired target region or selecting and using a part of a plurality of electrodes.

Accordingly, increased are needs for treatment devices adopting Cranial Electrotherapy Stimulation (CES) which are capable of applying stimulation to a desired target region accurately and appropriately.

### PRIOR ART DOCUMENT

### Patent Document

(Patent Document 1) Korean Patent Application Publication No. 10-2005-0030450

(Patent Document 2) Korean Patent Registration No. 10-0795830

(Patent Document 3) Korean Patent Registration No. 10-0938605

### SUMMARY OF THE INVENTION

Accordingly, the present invention is provided to solve the aforementioned drawbacks and aims to provide a body region dependent electric stimulation-based device for measuring biological signals. This measurement device measures a change in biological signals for a part of a user's body by an optimum biological measurement method among a plurality of biological signal measurement methods to reflect the change in the biological signals of the user's body accurately and transfers an optimum electric stimulation signal generated with reference of an optimum biological signal to a part of the user's body to maximize a treatment effect thereon.

Further, the present invention is provided to solve the aforementioned drawbacks and aims to provide an electric current stimulation device capable of selectively stimulating a region and a controlling method thereof. The electric current stimulation device is capable of intensively treating more precise region with an appropriate intensity of the current through a configuration that selects a treatment target region requiring stimulation depending on the type of the user's disease, optionally activates a plurality of electrodes for focusing microcurrents to the selected treatment target region and optionally determines the intensity of the microcurrent which the activated electrodes should induce and the route thereof, thereby providing the treatment target region with an environment for efficient treatment.

Meanwhile, technical aims to be achieved in the present invention are not limited to the aforementioned aims and other not-mentioned ones will be obviously understood by those killed in the art from the description below.

In order to achieve the aforementioned aims, a body region dependent electric stimulation-based device for measuring biological signals may include a plurality of measurement sensors which measures biological signals for a part of a user's body; a plurality of electric stimulation sensors which transfer a plurality of electric stimulation signals for treatment to the part of the user's body simultaneously or transferring the signals successively at a time interval; a monitoring portion which monitors biological signal based biorhythms of the user or the user's biorhythms changeable depending on the electric signals; database in which the user's biorhythms monitored in the monitoring portion are stored; an analysis portion in which a first analysis algorithm and a second algorithm were prestored, the first analysis algorithm analyzing information for the user's biorhythms and determines an optimum biological measurement sensor for measuring the optimum biological signal reflecting the user's biorhythms utmost, among the plurality of biological signal measurement sensors and the second analysis algorithm computing an optimum electric stimulation with reference to the optimum biological signal in order to treat a part of the user's body utmost; and a control portion which controls to measure the optimum biological signal from the biological signal measurement sensor and to generate the optimum electric stimulation signal from the electric stimulation sensors.

Further, a biological signal algorithm for measuring biological signals by at least one method of bioelectric signal measurement, bioimpedance signal measurement, biomagnetic signal measurement, biomechanical signal measurement and bioacoustic signal measurement may be prestored in the biological signal measurement sensor.

The first analysis algorithm may determine an optimum biological signal measurement method which compares a plurality of biological signals measured from the biological signal measurement sensor and measures a biological signal having relatively low or no unmeasured value or missing value among the plurality of biological signals.

Further, the control portion may control the biological signal measurement sensor to measure the optimum biological signal by the optimum biological signal measurement method.

Further, in the electric stimulation sensor, electric current stimulation algorithm may be prestored to generate stimulation signals by at least one electric stimulation method of interrupted galvanic current, alternating current, monophasic pulsed current, biphasic pulsed current, low frequency and near-infrared ray.

Further, the second algorithm may store a plurality of current stimulation signals to be generated by the electric stimulation method with reference to the optimum biological signal in the database. Following performing simulation in which the plurality of current stimulation signals to a virtual part of user's body that is the same region as the part of the user's body, this algorithm may calculate the optimum electric stimulation signal from the plurality of electric stimulation signals through the simulation result in order to treat the part of the user's body utmost.

Further, the control portion may control the electric stimulation sensor to generate and transfer the optimum electric stimulation signal to the part of the user's body.

Further, the body region dependent electric stimulation-based device for measuring biological signals may further include an information generation portion. The information generation portion measures biological signals for body regions of a plurality of targeted individuals to calculate average value of biological signals for respective regions of the targeted individuals, followed by modeling a virtual body of the user and adopting an average biological signal of the targeted individuals to each region of the virtual body of the user so as to generate and transmit information for the virtual body of the user to the database.

Further, the biological signal measurement sensor and the electric stimulation sensor may have a plurality of treatment sensors capable of performing operations in the optimum treatment mode which transfers the optimum electric stimulation signal to the part of the user's body while measuring biological signals or the optimum biological signal.

Further, in the plurality of treatment sensors, the respective treatment sensors may measure the optimum biological signal by the same biological signal measurement method or one of different biological signal measurement methods.

Further, the analysis portion may include: a third analysis algorithm which analyzes information for the user's biorhythm stored in the database and determines the optimum biological signal measurement method for measuring the optimum biological signal among biological signal measurement methods of the treatment sensor; a fourth analysis algorithm in which a plurality of current stimulation signals to be generated by the electric stimulation method with reference to the optimum biological signal may be stored in the database and following performing simulation in which the plurality of current stimulation signals is applied to a virtual part of user's body that is the same region as the part of the user's body, the optimum electric stimulation signal may be calculated from the plurality of electric stimulation signals through the simulation result in order to treat the part of the user's body utmost; and a fifth analysis algorithm which predicts a level of change in the biological signals for the virtual part of the user's body to determine the optimum treatment mode among a plurality of treatment modes when adopting the plurality of treatment modes prestored in the database.

Further, the control portion control to maintain the optimum treatment mode or to change other treatment modes before or while operating the treatment sensor in the optimum mode determined through the fifth analysis algorithm.

Further, the body region dependent electric stimulation-based device for measuring biological signals according to the present invention may further include a display that informs the user of the fifth analysis algorithm's determination result before the control portion controls the treatment sensor to be operated in the optimum treatment mode determined in the fifth analysis algorithm.

An electric current stimulation device capable of selectively stimulating a region according to the embodiment of the present invention as a means to achieve the aforementioned aims, may include a body; an electrode unit that is composed of a plurality of electrodes and is installed to the main body to contact with at least one part of a user's body; and a control unit that controls to transfer microcurrents of 1 mA or less to the user's brain by a Cranial Electrotherapy Stimulation (CES) method. The control unit may determine at least one stimulation area required be stimulated depending on the user's disease in the whole area of the brain and in order to treat the disease by focusing microcurrents on the stimulation area, may determine at least one of two or more of first electrodes required to be activated (ON) in the plurality of electrodes, an intensity of the microcurrent which respective electrodes of the first electrode should induce and a route thereof.

Further, the electric current stimulation device may further include a brain wave measurement unit that is installed to the main body to contact with the at least one part of the user's body and measure the user's brain wave (EEG, Electro Encephalo Graphy). After focusing the microcurrents on a first stimulation area preferentially determined, the control unit may determine a necessity to change the first stimulation area while continuously monitoring a state of the user's brain based on the brain wave measured in the brain wave measurement unit. When it is determined to change the first stimulation area to a new second stimulation area according to the determination result, in order to focus the microcurrent on the new second stimulation area, the control unit may change at least one of the first electrode, the intensity of the microcurrent which the respective electrodes of the first electrode should induce and the route thereof.

Further, at least one disease of sleep disorder, depression, dementia, Mild Cognitive Impairment (MCI), anxiety disorder, development disorder, Attention-Deficit/Hyperactivity Disorder (ADHD) may be treated by the CES method.

Further, the whole electrodes of the electric unit may be arranged that at least eight (8) or more electrodes make a circular form at regular intervals and the respective electrodes of the first electrode according to the arrangement may induce the microcurrents which are multi-crossable mutually by controlling of the control unit.

Further, the control unit may control the respective electrodes of the first electrode to be activated (ON) at the same time.

Further, the main body may be configured into a three-dimensional hemisphere shape having a circumference and to be wearable on the head of the user, and the whole of the electrodes of the electrode unit may be arranged along a circumference of the main body.

Further, a waveform of the microcurrent may be any one selected from DC, AC, Pulse (mono-phase, bi-phase, multi-phase), random waveform, coded waveform and waveform generated in an external device through wired/wireless connection.

Further, a pattern of the microcurrent may be any one selected from DC, sine, pulse, chirp, random patterns and a pattern generated in an external device through wired/wireless connection.

Further, in a case of the microcurrent is constant current, when passing the center of the stimulation area, the control unit may calculate the intensity of the microcurrent in a proportion of 100%, while calculating the intensity thereof in a proportion of 20% when passing outermost with reference to the stimulation area, so as to determine the intensity of the microcurrent.

Further, the control unit may be configured to allow controlling the intensity of the microcurrent while controlling voltage magnitude.

Further, the control unit may output different alarm signals when focusing the microcurrents on the stimulation area and when changing at least one of the first electrode, the intensity of the microcurrent which the respective electrodes of the first electrode should induce and the route thereof. The electric current stimulation device may further include an alarm unit which releases a preset pattern of sound and light according to alarm signals output from the control unit.

Further, the alarm unit may further include a sound output portion which releases a preset pattern of sound according to the alarm signals and acoustically informs the user of treatment in progress and changes in the brain wave; and a light emitting portion which releases a preset pattern of light according to the alarm signals and visually informs the user of treatment in progress and changes in the brain wave.

Further, the alarm unit may further include an insulation unit which insulates the electrode unit and the control unit with a circuit to prevent mutual electrical interference.

Further, the insulation unit may further include a first insulation portion which prevents electrical interference for a power signal; and a second insulation unit which prevents electrical interference for a control signal.

Further, the first insulation portion may be any one of a transformer or a DC-DC converter.

Further, the second insulation portion may be any one of a photo coupler or a signal transformer.

Meanwhile, a control method of an electric current stimulation device capable of selectively stimulating a region according to the embodiment of the present invention as a means to achieve the aforementioned aims, may include steps of: (a) measuring an arbitrary brain wave in real time by a brain wave measurement unit; (b) determining, by a control unit, at least one stimulation area to be stimulated in the whole area of the brain according to a user's disease on the basis of the brain wave measured in the step (a); (c) determining, by the control unit, at least one of two or more first electrodes required to be activated (ON) in the plurality of electrodes of an electrode unit, the intensity of the microcurrent which the respective electrodes of the first electrode should induce and the route thereof, in order to treat the disease by focusing microcurrents on the stimulation area; and (d) inducing, by the electrode unit, microcurrents resulting from the result determined in the step (c) and transferring the microcurrents to the user's brain by a CES method while focusing the microcurrents on the stimulation area determined in the step (b).

Further, the control method may further include a step of (e) changing at least one of the first electrode, the intensity of the microcurrent which the respective electrodes of the first electrode should induce and the route thereof, in order to focus the microcurrent on a new second stimulation area when the control unit determines a necessity to change preferentially determined first stimulation area while continuously monitoring a state of the user's brain based on the brain wave measured in the step (a) even after focusing the microcurrents on the first stimulation area preferentially determined in the step (d) and then determines to change the first stimulation area to the new second stimulation area according to the determination result.

Further, the control method may further include a step of (de-1) outputting, by the control unit, different alarm signals at the time of focusing the microcurrents on the stimulation area in the step (d) and of changing at least one of the first electrode, the intensity of the microcurrent which the respective electrodes of the first electrode should induce and the route thereof and releasing, by an alarm unit, a preset pattern of sound and light according to alarm signals output from the control unit.

### Advantageous Effects

According to the present invention, it is capable of accurately biological signals for a part of a user's body to increase treatment effect by measuring an optimum biological signal through a measurement method for measuring a plurality of biological signals.

Further, according to the present invention, it is capable of increasing treatment resulting in electric stimulation by generating the optimum electric stimulation signal from a plurality of electric stimulation methods and transferring the generated signal to the part of the user's body.

Further, according to the present invention, it is capable of maximizing treatment effects by treating the part of the user's body in the optimum treatment mode.

The present invention determines at least one stimulation area required to be stimulated according to the user's disease and is capable of intensively treating a more accurate area in an appropriate level through a control unit which determines at least one of two or more first electrodes required to be activated (ON) in a plurality of electrodes of an electrode unit, an intensity of the microcurrent which the respective electrodes of the first electrode should induce and the route thereof, in order to treat the disease by focusing microcurrents on the stimulation area, thereby allowing providing a more efficient treatment environment to the stimulation area.

Further, according to the present invention, the electrode unit is composed of a plurality of electrodes arranged into a circular form at regular intervals and generates a route for inducing microcurrents which are multi-crossable mutually as the respective electrodes are matched with each other to form a plurality of pairs, thereby securing focusing points for the microcurrent and intensity control ranges variously. Accordingly, it is capable of facilitating control of a stimulation intensity more simultaneously with responding to a wider range of the stimulation area.

In addition, the present invention is capable of inducing AI-based CES therapy through a configuration including a main body which is wearable on the head, a brain wave measurement unit which measures a brain wave in real time and a control unit which controls an electrode unit adaptively according to the measured brain wave, thereby achieving more effective treatment on at least one disease of sleep disorder, depression, dementia, Mild Cognitive Impairment (MCI), anxiety disorder, development disorder, Attention-Deficit/Hyperactivity Disorder (ADHD).

Further, according to the present invention, it is capable of preventing mutual electrical interference through an insulation unit which insulates the respective electrical configurations with a circuit, thereby allowing providing a more reliable treatment environment.

Furthermore, according to the present invention, it is capable of allowing the user to audiovisually recognize treatment in progress and changes in the brain wave easily through a configuration including a sound output portion which releases a preset pattern of sound according to the alarm signals and a light emitting portion which releases a preset pattern of light according to the alarm signals.

Meanwhile, effects to be achieved by the present invention are not limited to the aforementioned effects and other effects, which are not mentioned above, will be apparent to those skilled in the art from the following description.

### BRIEF DESCRIPTION OF THE DRAWINGS

The accompanying drawings in the specification illustrate an embodiment of the present invention. The technical essence of the present invention will be more clearly understood from the following detailed description taken in conjugation with the accompanying drawings. Therefore, the present invention will not be interpreted to be limited to the drawings in which:

FIG. 1 shows a block diagram representing a configuration of a region dependent electric stimulation-based device for measuring biological signals according to one embodiment of the present invention.

FIG. 2 shows block diagrams representing each configuration of a biological signal measurement sensor and an electric stimulation sensor respectively according to one embodiment of the present invention.

FIG. 3 shows block diagrams representing information prestored in each biological signal algorithm and electric stimulation algorithm respectively according to one embodiment of the present invention.

FIG. 4 shows a block diagram explaining that an analysis portion according to one embodiment of the present invention determines an optimum biological signal measurement sensor and calculates the optimum electric signal.

FIG. 5 shows a block diagram representing a configuration of a region dependent electric stimulation-based device for measuring biological signals according to one embodiment of the present invention.

FIG. 6 shows a block diagram showing a configuration of a treatment sensor according to another embodiment of the present invention.

FIG. 7 shows block diagrams representing information prestored in each biological signal algorithm and electric stimulation algorithm respectively according to another embodiment of the present invention.

FIG. 8 shows a block diagram explaining that an analysis portion according to another embodiment of the present invention determines the optimum biological signal measurement sensor, calculates an optimum electric signal and determines the optimum treatment mode.

FIG. 9 shows a block diagram schematically representing an electromagnetic configuration of an electric current stimulation device capable of selectively stimulating a region according to the embodiment of the present invention.

FIG. 10 shows a perspective drawing representing a main body and an alarm unit of the electric current stimulation device capable of selectively stimulating a region according to the embodiment of the present invention.

FIG. 11 shows a drawing schematically representing a circular arrangement of electrodes and routs of microcurrents resulting therefrom in an electric unit of the electric current stimulation device capable of selectively stimulating a region according to the embodiment of the present invention.

FIG. 12 shows a drawing schematically representing waveforms of the microcurrent between the electrodes in the electric unit of the electric current stimulation device capable of selectively stimulating a region according to the embodiment of the present invention.

FIG. 13 shows a drawing schematically representing electrical connection of the electric current stimulation device capable of selectively stimulating a region according to the embodiment of the present invention.

FIG. 14 shows a drawing schematically representing electrical connection of the electric current stimulation device capable of selectively stimulating a region according to another embodiment of the present invention.

FIG. 15 shows a flowchart showing the electric current stimulation device capable of selectively stimulating a region according to the embodiment of the present invention and a control method thereof.

### DETAILED DESCRIPTION OF THE EMBODIMENTS

Hereinafter, embodiments of the present invention will be described in detail and definitely referring to accompanying drawings, so that a person having ordinary skill in the art is able to implement the present invention easily. However, the description thereof explains the present invention structurally and functionally, and thus it should not be understood that described embodiments limit the patent scope of the present invention. That is, since it is allowable to modifying the embodiments variously, it should be understood that the patent scope of the present invention includes equivalents capable of implementing technical idea thereof. Further, since the aims and effects provided in the present invention do not mean that a specific embodiment should include the whole thereof or only them, it should not be understood that the patent scope of the present invention is limited thereto.

The terms used in the present invention should be understood as the followings.

Since the terms, such as “first”, “second”, etc., are used for distinguishing one element from other elements, the scope of the present invention should be not limited thereto. For example, “a first element” may be referred to as “a second element” and similarly hereto, “a second element” may be referred to as “a first element”. When mentioning that an element is “connected” to the other element, it may be connected directly thereto, however, it should be understood that there may be another element between them. Whereas, when mentioning that an element is “connected directly” to the other element, it should be understood that there may be not any other element between them. Meanwhile, it should be also understood in the same way as the above in case of expressions for explaining the relationship between elements, i.e., “between˜” and “directly between˜”, or “adjacent to˜” and “adjacent directly to˜”.

It should be understood that the singular expression includes the plural expression unless specifically stated otherwise. The terms, such as “comprise” and “have”, etc., indicate the existences of the implemented features, numbers, steps, operations, elements, components or any of combinations thereof. It should be understood that they do not preclude the potential existences or additions of one or more features, numbers, steps, operations, elements, components or any of combinations thereof.

Unless otherwise defined, all terms used herein have the same meanings as those commonly understood by those having ordinary knowledge in the art to which the present invention pertains. It should be understood that the terms defined in commonly used dictionaries, should be interpreted to be consistent with the meanings contextually stated in the field of relevant art and will not be interpreted to have idealized or excessively formalistic senses unless explicitly defined in the present invention.

### Region Dependent Electric Stimulation-Based Device for Measuring Biological Signals

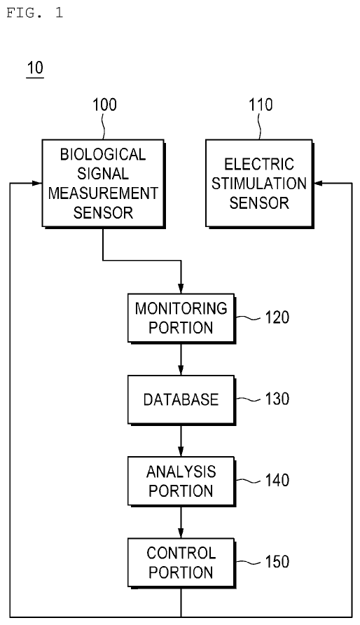

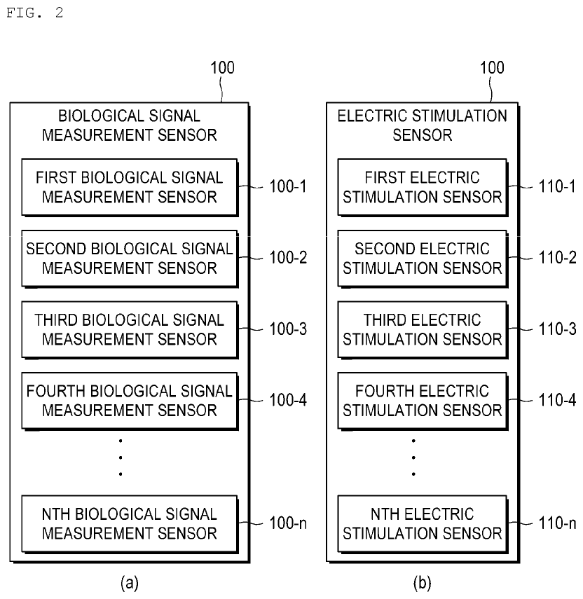

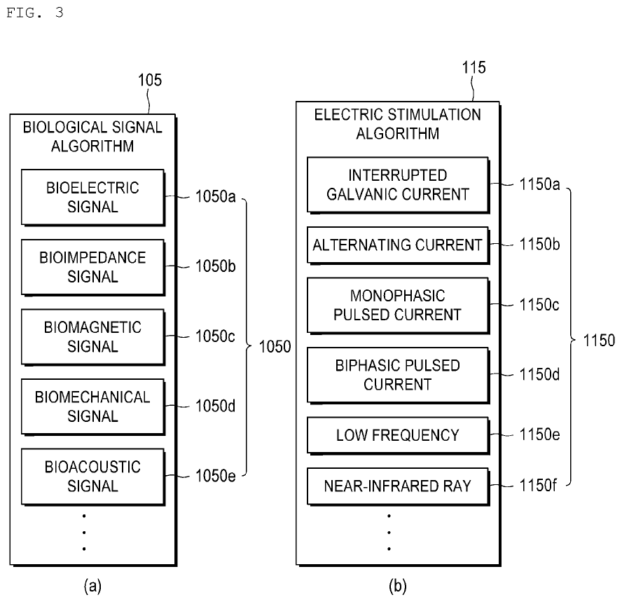

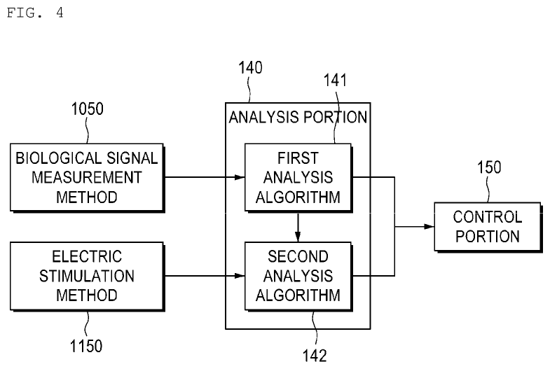

Hereinafter, a region dependent electric stimulation-based device for measuring biological signals 10 according to one embodiment of the present invention will be described in detail referring to FIG. 1 to FIG. 4.

The region dependent electric stimulation-based device for measuring biological signals 10 measures changes in biological signals for a part of a user's body to generate an optimum electric stimulation, followed by transferring an optimum electric signal allowing treating the part of the user's body utmost.

This region dependent electric stimulation-based device for measuring biological signals 10 includes a biological signal measurement sensor 100, an electric stimulation sensor 110, a monitoring portion 120, database 130, an analysis portion 140 and a control portion 150.

The biological signal measurement sensor 100 measure biological signals for the part of the user's body to determine an optimum biological signal measurement method in the analysis portion 140.

Herein, the part of the user's body means at least one body region of head, neck, abdomen, shoulders, arms, legs, waist, pelvises, hips, etc., and may mean two or more body regions.

This biological signal measurement sensor 100 may be composed of a plurality of biological signal measurement sensors 100-1, 100-2, 100-3, 100-4 . . . 100-n in order to either measure the biological signals for two or more body regions or to maximize measurement accuracy of the biological signal for the part of the user's body from a biological signal measurement area changeable depending on a circumference, length, volume, area of the user's body region.

Herein, each biological signal measurement sensor 100 composing the plurality of biological signal measurement sensors 100-1, 100-2, 100-3, 100-4 . . . 100-n may measure the biological signal for the part of the user's body by the same biological signal measurement method or other measurement methods different from each other.

The biological signal measurement sensor 100 may measure an optimum biological signal for the part of the user's body by the optimum biological signal measurement method determined in the analysis portion 140.

Herein, the optimum biological signal reflects the user's biorhythm relatively accurate in biological signals measurable by the plurality of biological signal measurement methods, meaning a signal having relatively low or no unmeasured value or missing value among the biological signals measured by the biological signal measurement sensor 100.

Further, in the biological signal measurement sensor 100, in order to measure the biological signal for the part of the user's body by the same biological signal measurement method or other measurement methods different from each other, prestored is a biological signal algorithm 105 which stores information for a plurality of biological signal measurement methods 1050 having different variables such as a biological signal processing method, etc.

As one particular example, the biological signal algorithm 105 may prestore information for a bioelectric signal measurement method 1050a, a bioimpedance signal measurement 1050b, a biomagnetic signal measurement method 1050c, a biomechanical signal measurement method 1050d and a bioacoustic signal measurement method 1050e.

Herein, the bioelectric signal measurement method 1050a may be a method for measuring an electrical signal, such as electrocardiogram, electroencephalogram, electrooculogram, electromyogram, etc., generated from the part of the user's body. The bioimpedance signal measurement method 1050b may be a method for measuring impedance by passing the microcurrent to the part of the user's body. The biomagnetic signal measurement method 1050c may be a method for measuring a minimal change in a magnetic signal (action current) generated from the part of the user's body. The biomechanical signal measurement method 1050d may be a method for measuring an epidemiological signal for the part of the user's body using a measurement means such as a transducer, etc. The bioacoustic signal measurement method 1050e may be a method for measuring an acoustic signal for the part of the user's body using a measurement means such as a stethoscope, a cardiophone, etc.

The biological signal algorithm 105 may additionally prestore information for other biological signal measurement methods (e.g., ballistocardiogram (BCG), photoplethysmogram (PPG), etc.) for measuring the biological signal for the part of the user's body, besides the information for the aforementioned biological signal measurement methods 1050a, 1050b, 1050c, 1050d, 1050e.

Accordingly, the biological signal measurement sensor 100 may measure the optimum biological signal for the part of the user's body by the biological signal measurement methods such as ballistocardiogram (BCG), photoplethysmogram (PPG), etc.

The electric stimulation sensor 110 transfers an electric stimulation signal to the part of the user's body by the same electric stimulation method or other electric stimulation methods different from each other through the electric stimulation method stored in the electric stimulation algorithm 115.

Herein, the electric stimulation signal is a microcurrent to be transferred to the part of the user's body and may a signal (energy) including microcurrent density (mA/Cm²), current period (usec) of the microcurrent and waveforms according to electric stimulation repetition period (Hz) resulting from the microcurrent.

This electric stimulation sensor 110 may transfer the optimum electric signal to the part of the user's body when the biological signal measurement sensor 100 measured the optimum biological signal and then the optimum electric stimulation signal was calculated from the analysis portion 140.

Herein, the optimum electric stimulation signal means an electric stimulation signal capable of maximizing a treatment effect on the part of the user's body. It is preferable that waveform thereof according to microcurrent density, current period, electric stimulation repetition period is changeable depending on the optimum biological signal measured from the biological signal measurement sensor 100.

Further, the electric stimulation sensor 110 may be composed of a plurality of electric stimulation sensors 110-1, 110-2, 110-3, 110-4 . . . 110-n in order to transfer the electric stimulation signal to two or more body regions or to transfer the optimum stimulation signal to an electric stimulation area changeable depending on the circumference, length, volume, area of the user's body region.

Herein, each electric stimulation sensor 110 composing the plurality of electric stimulation sensors 110-1, 110-2, 110-3, 110-4 . . . 110-n may transfer the optimum electric stimulation signal to the part of the user's boy simultaneously or successively at a time interval. Further, in the electric stimulation sensor 110, in order to generate the optimum electric stimulation signal in accordance with the optimum biological signal, prestored is an electric stimulation algorithm 115 which stores information for a plurality of electric stimulation methods 1150 having different variables such as an electric stimulation method and the type, waveform, pulse frequency, phase duration, pulse duration, interpulse interval of the electric current, etc.

As one particular example, the electric stimulation algorithm 115 may prestore information for an interrupted galvanic current method 1150a, an alternating current method 1150b, a monophasic pulsed current method 1150c, a biphasic pulsed current method 1150d, a low frequency method 1150e and a near-infrared ray method 1150f.

The electric stimulation algorithm 115 may additionally prestore information for other electric stimulation methods (e.g., mid frequency, high frequency, etc.) having waveforms different from those of the aforementioned electric stimulation methods (1150a, 1150b, 1150c, 1150d, 1150e, 1150f), besides the information for the methods (1150a, 1150b, 1150c, 1150d, 1150e, 1150f).

Further, the electric stimulation algorithm 115 may additionally prestore information for other electric stimulation methods (e.g., TDCS, DBS, TACS, ECT, ECS, CES, NIR, etc.) different from the aforementioned electric stimulation methods (1150a, 1150b, 1150c, 1150d, 1150e, 1150f), besides the information for the methods (1150a, 1150b, 1150c, 1150d, 1150e, 1150f).

Accordingly, the electric stimulation sensor 110 may transfer an optimum electric stimulation for the part of the user's body by the electric stimulation methods such as mid frequency, high frequency, TDCS, DBS, TACS, ECT, ECS, CES, NIR, etc.

The monitoring portion 120 is connected with the biological signal measurement sensor 100 to monitor the user's biorhythm based on the biological signal measured in the biological signal measurement sensor 100, or the user's biorhythm changeable as the electric stimulation sensor 110 transfers the optimum electric stimulation signal to the part of the user's body.

As the biological signal measurement sensor 100 is configured with several biological signal measurement sensors, this monitoring portion 120 may separately monitor the user's biorhythms based on the biological signal measured from the respective biological signal measurement sensors 100.

Further, the monitoring portion 120 may separately monitor a plurality of the user's biorhythms resulting from a plurality of the biological signal measurement methods 1050, when the respective biological signal measurement sensors 100 measure the biological signals by the biological signal measurement methods 1050 different from each other.

As one particular example, when the first biological signal measurement sensor 100-1 and the second biological signal measurement sensor 100-2 measure the biological signals by the bioelectric signal measurement method 1050a and the bioimpedance signal measurement method 1050b respectively, the monitoring portion 120 may monitor each of the user's biorhythms resulting from the bioelectric signal measurement method 1050a and the bioimpedance signal measurement method 1050b respectively, as dividing the first biological signal measurement sensor 100-1 and the second biological signal measurement sensor 100-2.

Further, when the biological signal measurement sensor 100 measures the optimum biological signal from the part of the user's body by the optimum biological signal measurement method, the monitoring portion 120 may monitor the user's biorhythm based on the optimum biological signal measured from the biological signal measurement sensor 100.

In the database 130, stored is information for the user's biorhythm monitored in the monitoring portion 120.

Herein, the information of the user's biorhythm stored in the database 130 may be the biological signal measured from the plurality of biological signal measurement sensors 100 or the optimum biological signal.

Further, in the database 130, a program for controlling the region dependent electric stimulation-based device for measuring biological signals 10 of the present invention may be prestored as well as the information for the user's biorhythm.

The analysis portion 140 determines the optimum biological signal measurement sensor for measuring the optimum biological signal by analyzing the information for the user's biorhythm stored in the database 130 and a first analysis algorithm 141 and a second analysis algorithm 142 are prestored therein to calculate the optimum electric stimulation signal to be transferred to the part of the user's body.

The first analysis algorithm 141 compares the plurality of biological signals measured from the plurality of biological signal measurement sensors 100 to determine, as the optimum biological signal measurement method, the biological signal measurement method which measures a biological signal having relatively low or no unmeasured value or missing value among the plurality of biological signals.

As one particular example, when the first biological signal measurement sensor 100-1 and the second biological signal measurement sensor 100-2 measure the biological signals by the bioelectric signal measurement method 1050a and the bioimpedance signal measurement method 1050b respectively, the first analysis algorithm 141 compares the biological signal of the first biological signal measurement sensor 100-1 and the biological signal of the second biological signal measurement sensor 100-2, stored in the database 130 to deduce a conclusion that the biological signal of the first biological signal measurement sensor 100-1 has relatively low or no unmeasured value or missing value as a result, thus allowing determining the bioelectric signal measurement method 1050a as the optimum biological signal measurement method.

When the biological signal measurement sensor 100 measures the optimum biological signal for the part of the user's body by the optimum biological signal measurement method determined from the first analysis algorithm 141, the second analysis algorithm 142 performs simulation in which a plurality of electric stimulation signals to be generated by the plurality of electric stimulation methods 1150 with reference to the optimum biological signal is applied to a virtual part of user's body that is the same region as the part of the user's body, followed by calculating the optimum electric stimulation signal from the plurality of electric stimulation signals through the simulation result in order to treat the part of the user's body utmost.

As one particular example, when the second analysis algorithm 142 performs simulation in which a respective plurality of electric stimulation signals to be generated by the interrupted galvanic current method 1150a, the alternating current method 1150b, the monophasic pulsed current method 1150c, the biphasic pulsed current method 1150d, the low frequency method 1150e and the near-infrared ray method 1150f with reference to the optimum biological signal is applied to the virtual part of user's body that is the same region as the part of the user's body, followed by determining the electric stimulation signal generated by the interrupted galvanic current method 1150a as the optimum electric stimulation signal for treating the part of the user's body utmost, the electric stimulation signal of the interrupted galvanic current method 1150a may be calculated as the optimum electric stimulation.

Herein, the information for the virtual part of the user's body is information generated by measuring biological signals of targeted individuals by an information generation portion (not illustrated) to calculate average value of biological signals for respective regions of the targeted individuals, followed by modeling a virtual body of the user and adopting an average biological signal of the targeted individuals to each region of the virtual body of the user. The information may be transferred to the database 130 by the information generation portion (not illustrated).

Accordingly, it is preferable that the second analysis algorithm 142 loads the information for the part of the user's body from the database 130 whenever simulation is made.

Further, since the optimum biological signal may be measured or the part of the user's body which the optimum electric stimulation signal is transferred to may be changeable, it is preferable to prestore the information for the user's virtual body that is the same as all region of the user's body in the database 130.

When the optimum biological signal is determined by the first analysis algorithm 141, the control portion 150 controls the biological signal measurement sensor 100 to measure the optimum biological signal for the part of the user's body by the optimum biological signal measurement method.

Further, when the optimum electric stimulation signal is calculated by the second analysis algorithm 142, the control portion 150 controls the electric stimulation sensor 110 to generate the optimum electric stimulation signal, allowing transferring the optimum electric stimulation signal to the part of the user's body from the electric stimulation sensor 110.

Further, the control portion 150 may control overall operation process of the region dependent electric stimulation-based device for measuring biological signals 10 according to the present invention.

As one particular example, the control portion 150 may control: the process for measuring the biological signal or optimum biological signal in the biological signal measurement sensor 100; the process for generating the optimum electric stimulation signal in the electric stimulation sensor 110; the process for monitoring the user's biorhythm in the monitoring portion 120; the processes for storing the information for the user's biorhythm and for transmitting the information for the virtual part of the user's body in the database 130; and the processes for determining the optimum biological signal measurement method and for calculating the optimum electric stimulation signal in the analysis portion 140.

Meanwhile, the region dependent electric stimulation-based device for measuring biological signals 10 according to the present invention may further include a power supply portion (not illustrated) which supplies electric power for operating the biological signal measurement sensor 100, the electric stimulation sensor 110, the monitoring portion 12, the database 130 and the analysis portion 140.

Further, the region dependent electric stimulation-based device for measuring biological signals 10 according to the present invention may further include a first alarm portion (not illustrated) which generates a first colored light and a second colored light for a predetermined period of time in any one way of emitting light, flashing light and emitting/flashing light to inform the user of the optimum biological signal measurement.

As one particular example, when the optimum biological signal is measured, the first alarm (not illustrated) may emit the first colored light for 1 to 4 seconds, while flashing the second colored light for 5 to 10 seconds when the optimum biological signal is not measured.

As mentioned above, the first alarm portion (not illustrated) may be preset to differentiate each timing for generating the first and second colored lights respectively according to the optimum biological signal measurement or this may be controlled by the control portion (150).

Further, the region dependent electric stimulation-based device for measuring biological signals 10 according to the present invention may further include a second alarm portion (not illustrated) which generates first and second sounds of which intensity (or amplitude), pitch (or frequency), timbre (or wave) are different each other for a predetermined period of time in order to inform the user the optimum biological signal measurement.

As one particular example, when the optimum electric stimulation signal is generated, the second alarm portion (not illustrated) may generate a first sound for 1 to 2 seconds, while generating a second sound for 3 to 5 seconds when the optimum electric stimulation signal is not generated.

As mentioned above, the second alarm portion (not illustrated) may be preset to differentiate each method and timing for generating the first and second sounds respectively, or this may be controlled by the control portion 150.

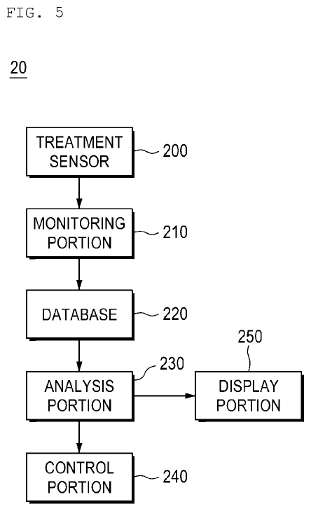

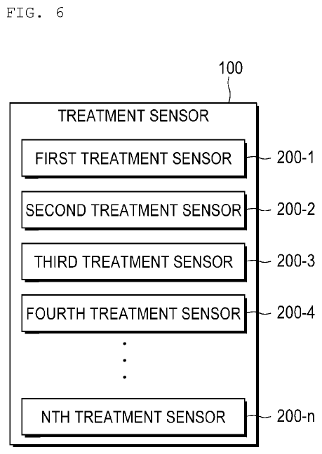

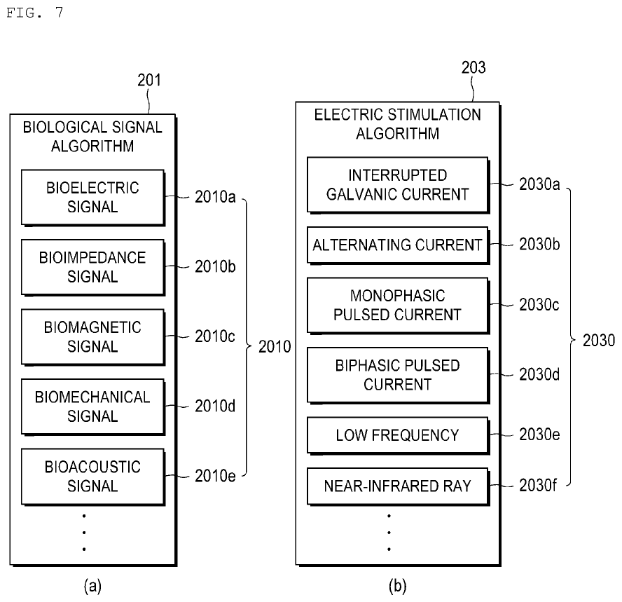

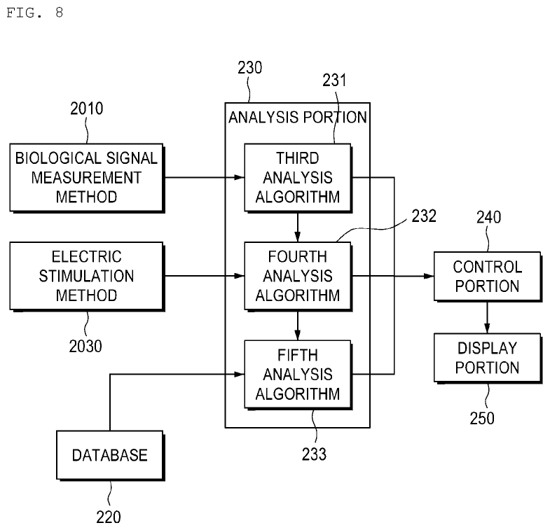

Hereinafter, referring to FIG. 5 to FIG. 8, a region dependent electric stimulation-based device for measuring biological signals 20 according to another embodiment of the present invention will be described in detail.

The region dependent electric stimulation-based device for measuring biological signals 20 according to another embodiment of the present invention is an example for modifying the aforementioned embodiment of the region dependent electric stimulation-based device for measuring biological signals 10. For brevity of explanation, description for the same configuration elements as the aforementioned embodiment will be omitted even though numerical references are different from each other.

The region dependent electric stimulation-based device for measuring biological signals 20 includes a treatment sensor 200, a monitoring portion 210, a database 220, an analysis portion 230, a control portion 240 and a display 250.

The treatment sensor 200 is a hybrid sensor having the biological signal measurement sensor 100 and the electric stimulation sensor 110, allowing transferring the optimum electric stimulation signal to the part of the user's body while measuring the biological signal for the part of the user's body or the optimum biological signal.

This treatment sensor 200 may be composed of a plurality of treatment sensors 200-1, 200-2, 200-3, 200-4 . . . 200-n in order to either measure the biological signals for two or more body regions or to maximize measurement accuracy of the biological signal for the part of the user's body from a biological signal measurement area changeable depending on a circumference, length, volume, area of the user's body region. Alternatively, this may transfer the optimum electric stimulation signal to either two or more body regions or transfer the biological signal measurement area changeable depending on a circumference, length, volume, area of the user's body region.

Herein, each treatment sensor 200 composing the plurality of treatment sensors 200-1, 200-2, 200-3, 200-4 . . . 200-n may measure the biological signal for the part of the user's body by the same biological signal measurement method or other measurement methods different from each other and transfer the optimum electric stimulation signal for the part of the user's body simultaneously or successively at a time interval.

Further, in the treatment sensor 200, in order to measure the biological signal for the part of the user's body by the same biological signal measurement method or other measurement methods different from each other, prestored is a biological signal algorithm 201 which stores information for a plurality of biological signal measurement methods 2010 having different variables such as a biological signal processing method, etc.

Since information for the biological signal measurement method 2010 prestored in the biological signal algorithm 201 is the same as the biological signal measurement method 1050 of the aforementioned embodiment but different only in numerical references, the detailed description thereof will be omitted hereinafter for convenience of explanation.

Further, in the treatment sensor 200, in order to generate the optimum electric stimulation signal in accordance with the optimum biological signal, prestored is an electric stimulation algorithm 203 which stores information for a plurality of electric stimulation methods having different variables such as an electric stimulation method and the type, waveform, pulse frequency, phase duration, pulse duration, interpulse interval of the current, etc.

Since information for the electric stimulation method 2030 prestored in the electric stimulation algorithm 203 is the same as the electric stimulation method 1150 of the aforementioned embodiment but different only in numerical references, the detailed description thereof will be omitted hereinafter for convenience of explanation.

As compared to the monitoring portion 120 of the aforementioned embodiment, the monitoring portion 210 is modified by replacing the biological signal measurement sensor 100 with the treatment sensor 200 but the monitoring process is the same as the aforementioned embodiment. Thus, the detailed description thereof will be omitted hereinafter for convenience of explanation.

The database 220 may store the information for the use's biorhythm monitored in the monitoring portion 220. Further, this may prestore a program for controlling the region dependent electric stimulation-based device for measuring biological signals 20 of the present invention, a treatment mode of the treatment sensor 200 and information for a virtual part of the user's body.

Herein, the treatment mode may be an instruction mode to operate the treatment sensor 200 by the one biological signal measurement method 2010 and the one electric stimulation method 2030.

The analysis portion 230 may prestore, a third analysis algorithm 231 which analyzes the information for the user's biorhythm stored in the database 220 to determine the optimum biological measurement method and a fourth analysis algorithm 231 which calculates the optimum electric stimulation signal to be transferred to the part of the user's body.

The third algorithm 231 may determine the biological signal measurement method which measures a biological signal having relatively low or no unmeasured value or missing value among the plurality of biological signals as the optimum biological signal measurement method by comparing the plurality of biological signals measured in the plurality of treatment sensors 200.

When the treatment sensor 200 measures the optimum biological signal for the part of the user's body by the optimum biological measurement method determined in the third analysis algorithm 231, the fourth analysis algorithm 232 performs simulation in which a plurality of electric stimulation signals to be generated by the plurality of electric stimulation methods 2030 with reference to the optimum biological signal is applied to the virtual part of user's body that is the same region as the part of the user's body, followed by calculating the optimum electric stimulation signal from the plurality of electric stimulation signals through the simulation result in order to treat the part of the user's body utmost.

The control portion 240 may control to maintain the optimum treatment mode or to change other treatment modes before or while operating the treatment sensor 200 in the optimum treatment mode.

Herein, the optimum treatment mode may be an instruction mode to operate the treatment sensor 200 by the optimum biological signal measurement method 2010 and the optimum electric stimulation method 2030 in order for treating the part of the user's body utmost.

Meanwhile, the analysis portion 230 may prestore a fifth analysis algorithm 233 to determine the optimum treatment mode.

When a plurality of treatment modes prestored in the data base 220 is adopted, the fifth analysis algorithm 233 predicts a level of change in the biological signals (user's biorhythms) for the virtual part of the user's body to determine the optimum treatment mode among the plurality of treatment modes.

The control portion 240 may control the treatment sensor 200 to be operated in the optimum treatment mode determined in the fifth analysis algorithm 233.

The display portion 250 may inform the user of the determination result of the fifth analysis algorithm 233 before the treatment sensor 200 is operated in the optimum treatment mode by the control portion 240.

Meanwhile, the region dependent electric stimulation-based device for measuring biological signals according to another embodiment of the present invention may have a power supply portion (not illustrated), a first alarm portion (not illustrated) and a second alarm portion (not illustrated) similarly to the aforementioned embodiment. Since those power supply portion (not illustrated), first alarm portion (not illustrated) and second alarm portion (not illustrated) are the same as the aforementioned embodiment, the detailed description thereof will be omitted hereinafter for convenience of explanation.

### Electric Current Stimulation Device Capable of Selectively Stimulating a Region and Control Method Thereof

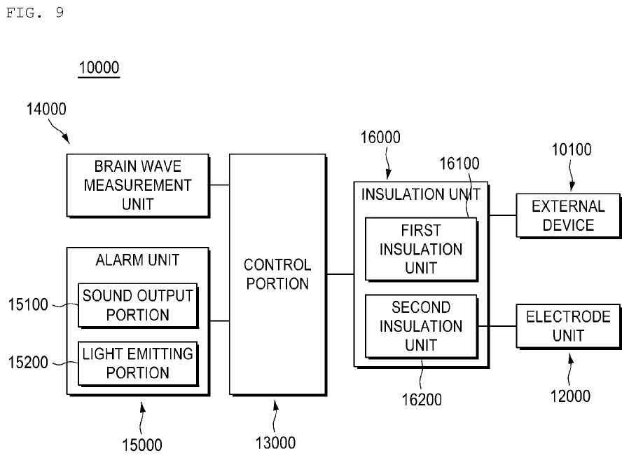

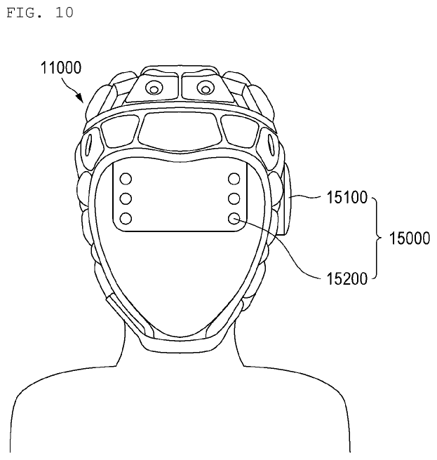

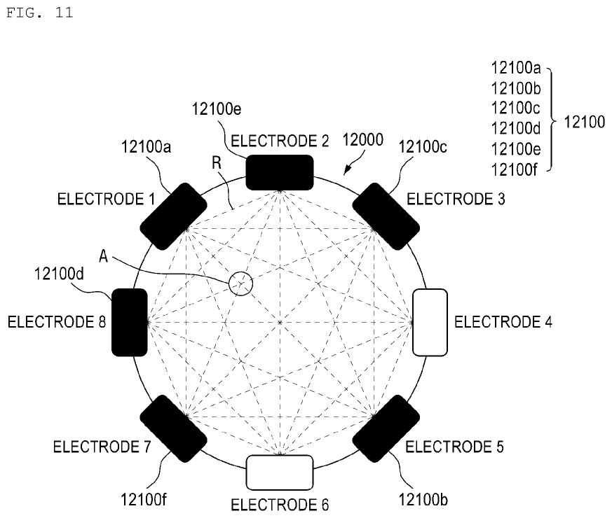

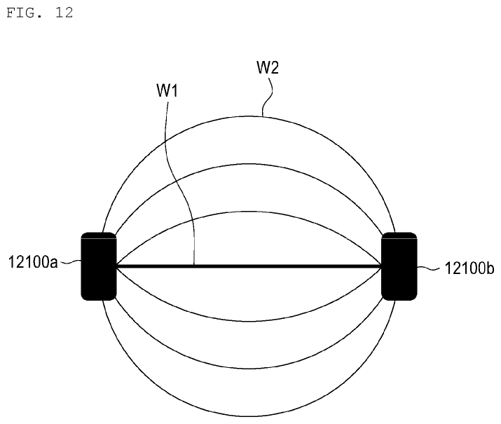

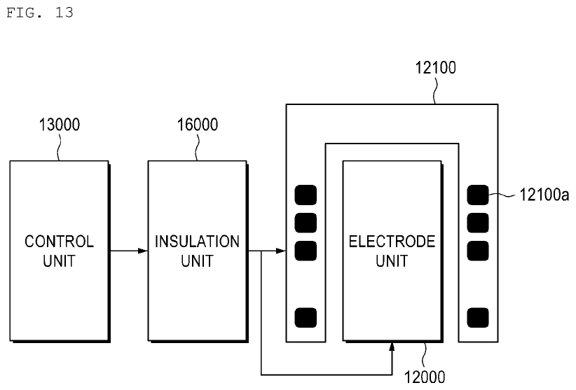

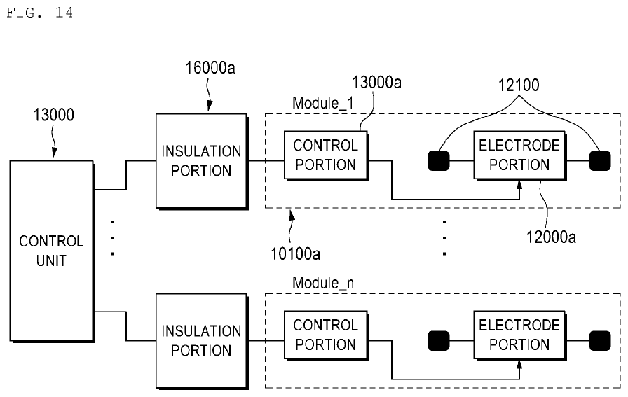

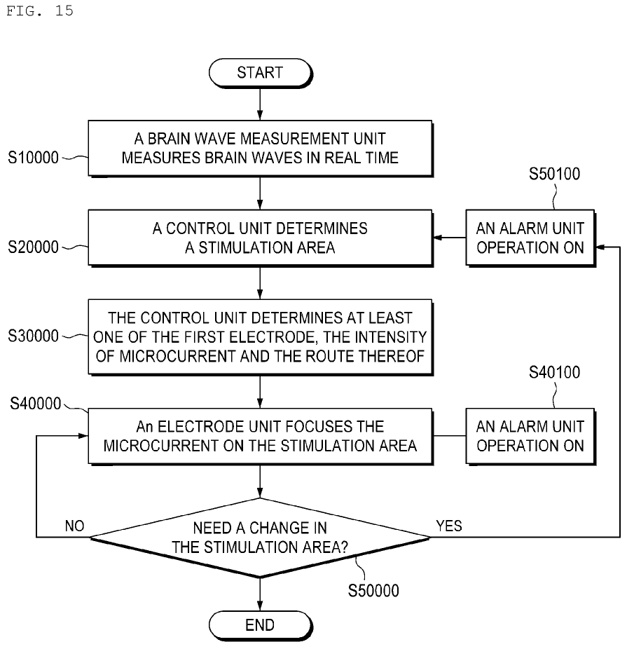

FIG. 9 shows a block diagram schematically representing an electromagnetic configuration of an electric current stimulation device capable of selectively stimulating a region according to the embodiment of the present invention. FIG. 10 shows a perspective drawing representing a body and an alarm unit of the electric current stimulation device capable of selectively stimulating a region according to the embodiment of the present invention. FIG. 11 shows a drawing schematically representing a circular arrangement of electrodes and routs of microcurrents resulting therefrom in an electric unit of the electric current stimulation device capable of selectively stimulating a region according to the embodiment of the present invention. FIG. 12 shows a drawing schematically representing waveforms of the microcurrent between the electrodes in the electric unit of the electric current stimulation device capable of selectively stimulating a region according to the embodiment of the present invention. FIG. 13 shows a drawing schematically representing electrical connection of the electric current stimulation device capable of selectively stimulating a region according to the embodiment of the present invention. FIG. 14 shows a drawing schematically representing electrical connection of the electric current stimulation device capable of selectively stimulating a region according to another embodiment of the present invention. FIG. 15 shows a flowchart showing the electric current stimulation device capable of selectively stimulating a region according to the embodiment of the present invention and a control method thereof.

As shown in FIG. 9 to FIG. 13, the electric current stimulation device capable of selectively stimulating a region 10000 according to the present invention is a medical instrument which treats at least a part of the user's body using the microcurrents and may be configured to include a main body 11000, an electrode unit 12000, a control unit 13000, a brain wave measurement unit 14000, an alarm unit 15000 and an insulation unit 16000.

The main body 11000 covers respective configuration elements of the present invention overall. Preferably, this may be configured into a three-dimensional hemisphere shape having a circumference and to be wearable on the head of the user's body.

This main body 11000 may be a headgear shaped one referring to FIG. 10 but is not limited thereto. The main body may be fabricated into various shapes by those skilled in the art and various materials may be used therefor.

The electrode unit 12000 is composed of a plurality of electrodes and installed in the main body 11000 to contact to at least a part of the user's body, this meaning an assembly of the respective electrodes.

Preferably, this electrode unit 12000 is equipped along the configuration of the aforementioned main body 11000 allowing contacting to the head of the user's body. More preferably, at least eleven or more electrode units may be arranged into a circular form at regular intervals along the circumference of the main body 11000. In FIG. 10 according to the present invention, the electrode unit is a built-in part of the main body and thus not illustrated.

That is, the electrode is configured to indirectly contact with the head of the user's body, allowing transferring microcurrents of 1 mA or less to the brain by electrical joint between configuration elements to be described hereinafter. Consequently, this provides an environment capable of inducing a treatment method of Cranial Electrotherapy Stimulation (CES).

Further, the electrode unit 12000 may be configured to include a stimulation generation portion (not illustrated) which generates electric stimulation for inducing the microcurrents.

The stimulation generation portion may be supplied with electric power by a separate power supply portion (not illustrated) and may be configured to generate electric stimulation for inducing the microcurrent according to the control of the control unit 13000 to transfer the generated electric stimulation to the electrode.

Herein, the electric stimulation means at least one of microcurrent density per unit area (mA/Cm²) by the control of the control unit 13000, current period (usec) to flow the microcurrent and microcurrent wave forms according to repetition period (Hz) of the microcurrent stimulation.

Further, the power supply portion supplies electric power to the stimulation generation portion of the aforementioned electrode unit 12000. According to a preferable embodiment of the present invention, taking efficiency into account, this may be configured to perform ON/OFF operations by the control of the control unit 13000, and thus selectively supplies electric power to the stimulation generation portion by the control unit 13000.

Since technique for supply electric power of the power supply portion is a publicly known technique, the description thereof is omitted in the drawing of the present invention. Further, it is available for those skilled in the art to adopt various types of publicly known power supply devices (modules) within the technical scope of the present invention.

The control unit 13000 controls the operation of the power supply portion controls the operation of the power supply portion and further controls the electrode unit 12000 to transfer microcurrents of 1 mA or less to the user's brain by the CES method. This may be configured to adaptively control the electrode unit 12000 according to the brain wave measured in the brain wave measurement unit 14000.

In more particularly, according to the present invention, the control unit 13000 may be configured to determine at least one stimulation area A required to be stimulated according to the user's disease from the whole area of the brain.

Further, in order to treat the disease by focusing the microcurrents on the stimulation area A, it is preferable to configure the control unit 13000 to determine at least one of at least two or more first electrodes 12100 to be activated (ON) in the plurality of the electrodes, the intensity of the microcurrent which respective electrodes 12100a, 12100b, 12100c, 12100d, 12100e, 12100f of the first electrode 12100 should induce and the route thereof.

For this, the control unit 13000 may preset each stimulation pattern information corresponding to the user's brain related diseases respectively.

Herein, according to the preferable embodiment of the present invention, the disease is preferably at least one of sleep disorder, depression, dementia, Mild Cognitive Impairment (MCI), anxiety disorder, development disorder, Attention-Deficit/Hyperactivity Disorder (ADHD).

For example, each of these diseases has a different stimulation area required to be stimulated on the brain and the stimulation intensity therefor is also different.

The stimulation pattern information corresponds to the above-listed diseases, respectively. For the brain wave measured in the brain wave measurement unit 14000, the stimulation pattern information may include information for that what and how many electrodes should be selected (ON) and how many pairs of the electrodes should be matched (or linked) 12100a-12100b, 12100c-12100d, 12100e-12100f (See FIG. 11), information on what waveforms (W1 or W2) microcurrents between the matched electrodes 12100a-12100b, 12100c-12100d, 12100e-12100f should have (See FIG. 12), and information to what extent voltage should be controlled to taking account into impedance for the microcurrents between the matched electrodes 12100a-12100b, 12100c-12100d, 12100e-12100f.

That is, the control unit 13000 matches and analyzes the preset stimulation pattern information and the brain wave measured in the brain wave measurement unit 14000. According to the analysis result, determined are the stimulation area A, the first electrode 12100 for the stimulation area A, the intensity of the microcurrent for the first electrode 12100, etc. and the route thereof.

Meanwhile, the embodiment of the present invention applies, as a signal for controlling the electrode unit 12000, only the brain wave of the brain wave measurement unit 1400 to the control unit 13000. However, the control unit 13000 may be configured to additionally receive an arbitrary stimulation demand signal for controlling the electrode unit 12000.

This stimulation demand signal is an electric signal for the requirement for changing the microcurrents to those preferable to the user or intentionally desired by the user. This may be manually input by the user through the input means of an external device 10100 separately provided or automatically input through various types of publicly known sensor units (not illustrated) within the technical scope of the present invention allowing sensing the user's brain state.

For example, the sensor unit may be configured to include a voice sensor which interlocks with the control unit 13000 and on which a voice cognitive SDK is mounted. In a case of this configuration, the stimulation demand signal may be input through the user's voice.

The brain wave measurement unit 14000 is a sensor which is equipped to the main body 11000 to contact with at least the part of the user's body so as to measure the brain wave of the user (EEG, Electro Encephalo Graphy). More preferably, this may be configured to contact to the user's head.

Herein, the brain wave measurement unit 14000 may be applied to various types of publicly known brain wave sensors, and this may be implemented through a separate electrode for sensing brain wave (not illustrated) and the detailed description thereof will be omitted hereinafter.

Consequently, the control unit 13000 according to the preferable embodiment of the present invention may be configured to analyze the brain wave measured in the brain wave measurement unit 14000 in real time to determine the stimulation area A according to the type of the brain diseases and to determine at least one of the first electrode 12100 corresponding to the determined stimulation area A, the intensity of the microcurrent which the respective electrodes 12100a, 12100b, 12100c, 12100d, 12100c, 12100f of the first electrode 12100 should induce and the route thereof.

At this time, the first electrode 12100 of the electrode unit 12000 includes the electrodes 12100a, 12100b, 12100c, 12100d, 12100c, 12100f selected by the control of the control unit 13000. According to the control of the control unit 13000, the plurality of electrodes is selected and activated (ON) to induce the microcurrent. In particular, a these are multi-crossably matched to be the plurality of pairs of different electrodes 12100a-12100b, 12100c-12100d, 12100e-12100f and may induce the microcurrents which are focused on the stimulation area A.

The whole configuration of this electrode unit 12000, more preferably, is composed of eight electrodes and may induce the microcurrents having various routes (R) as shown in FIG. 11. However, this is not limited thereto and is preferably composed of eight or more electrodes so as to secure more various induction routes of the microcurrents.

Herein, according to the present invention, it is preferable that the control unit 13000 controls the respective electrodes 12100a, 12100b, 12100c, 12100d, 12100c, 12100f of the first electrode 12100 to be activated (ON) at the same time. Accordingly, it may be achieved to focus the microcurrents on the stimulation area A at the same time.

At this time, the wave form of the microcurrents induced in the respective electrodes 12100a, 12100b, 12100c, 12100d, 12100c, 12100f is preferably a (mono phase, bi-phase, multi-phase) pulse. However, this may be any one of DC, AC, random waveform, coded waveform and waveform generated in an external device 10100 through wired/wireless connection, and is not limited thereto.

At the same time, the pattern of the microcurrents induced in the respective electrodes 12100a, 12100b, 12100c, 12100d, 12100c, 12100f is preferably a regular pattern such as DC, sine, pulse, chirp, etc. However, this may be also a random pattern and a pattern generated in an external device 10100 through wired/wireless connection but is not limited thereto.

For example, according to the control unit 13000 of the present invention, in a case that the microcurrent between the electrodes 12100a, 12100b to be matched is constant current, it is preferable to determine an intensity of the microcurrent by calculating in a proportion of 100% when a wave form is a first wave form W1 passing the center with respect to the stimulation area A (FIG. 11).

On the other hand, in a case that that the microcurrent between the electrodes 12100a, 12100b to be matched is constant current, an intensity of the microcurrent may be determined by calculating in a proportion of 20% when a wave form is a second wave form W2 passing outermost with reference to the stimulation area A (FIG. 11). Furthermore, an intensity of the microcurrent may be determined by calculating in a proportion of any one value therebetween when a wave form is an arbitrary wave form positioning between the first and second wave forms W1, W2 (not illustrated).

It is preferable to configure such an intensity of the microcurrent to be controllable while the control unit 13000 controls voltage taking account into impedance for the microcurrents between the respective electrodes 12100a-12100b, 12100c-12100d, 12100e-12100f of the first electrode 12100. However, it is not limited thereto and may be designed through various modifications by those skilled in the art.

Meanwhile, according to the preferable embodiment of the present invention, after focusing the microcurrents on a first stimulation area preferentially determined (numerical reference not indicated) in the aforementioned stimulation area A, the control unit 13000 determine a necessity to change the first stimulation area while continuously monitoring a state of the user's brain on the basis of the brain wave measured in the brain wave measurement unit 14000.

Further, when it is determined to change the first stimulation area to a new second stimulation area in the stimulation area A according to the determination result, in order to focus the microcurrent on the new second stimulation area (numerical reference not indicated), it is preferable to configure that the control unit 13000 may change at least one of the first electrode, the intensity of the microcurrent which the respective electrodes of the first electrode should induce and the route thereof.

Furthermore, in each case of focusing the microcurrents on the stimulation area A and changing at least one of the first electrode 12100, the intensity of the microcurrent which the respective electrodes 12100a, 12100b, 12100c, 12100d, 12100e, 12100f of the first electrode 12100 should induce and the route thereof, the control unit 13000 may be configured to output different alarm signals, respectively.

For example, the former case of focusing the microcurrents on the stimulation area A may mean that treatment is in progress through the electric current stimulation device. The latter case of changing at least one of the first electrode 12100, the intensity the microcurrent which the respective electrodes 12100a, 12100b, 12100c, 12100d, 12100e, 12100f of the first electrode 12100 should induce and the route thereof may mean that there is a change in the brain wave of the user.

In this regard, the alarm unit 15000 may be configured to release a preset pattern of sound and light according to alarm signals output from the control unit 13000.

This alarm unit 15000 performs functions which allow the user to recognize treatment in progress and changes in the brain wave (brain state) and may be configured to include a sound output portion 15100 and a light emitting portion 15200.

The sound output portion 15100 releases a preset pattern of sound according to the aforementioned alarm signals and performs a function acoustically informing the user of treatment in progress and changes in the brain wave.

This sound output portion 15100 may be implemented by setting sound pattern information such as sound intensity, pitch, timbre, etc., differently according to the respective alarm signals and various types of publicly known speaker modules may be adopted thereto.

The light emitting portion 15200 releases a preset pattern of light according to the aforementioned alarm signals and performs a function visually informing the user of treatment in progress through the electric current stimulation device 10000 and changes in the brain wave.

This light emitting portion 15200 may be implemented by setting lighting pattern information such as color, flash, etc., differently according to the respective alarm signals. It is preferable to adopt LED thereto, however, it is not limited thereto. Various types of publicly known lighting modules may be adopted by those skilled in the art.

In addition, in the preferable embodiment of the present invention, the alarm unit 15000 adopts the light emitting portion 15200 as a configuration element for the acoustic alarm. However, it is not limited thereto and may be configured with a similar spectrum of configuration elements. This may be configured to further include or replaced with a display portion (not illustrated) which visually represents preset texts and images on an arbitrary screen.

This display portion may be configured to include at least one of a liquid crystal display (LCD), a thin film transistor-liquid crystal display (TFT CLCD), an organic light-emitting diode (OLED), a flexible display and a 3D display.

The insulation unit 16100 is a configuration element which insulates the aforementioned electrode unit 12000, the power supply unit and the control unit 13000 with a circuit to perform a function preventing mutual electrical interference. In particular, this may be configured to include a first insulation portion 16100 and a second insulation portion 16200.

The first insulation portion 16100 insulates the power supply portion with a circuit to prevent electrical interference for a power signal.

The first insulation portion 16100 is preferably a transformer but a DC-DC converter is also applicable. However, it is not limited thereto.

The second insulation portion 16200 insulates the electrodes of the electrode units 12000, and the stimulation generation portion and the control unit 130000 with a circuit to perform a function prevention electrical interference for a control signal.

Herein, the second insulation portion 16200 is preferably a photo coupler but a signal transformer is also applicable. However, it is not limited thereto and may be designed through various modifications by those skilled in the art within the technical scope of the present invention.

Meanwhile, as another embodiment of the present invention, the electrode unit 12000 and the control unit 13000 may be configured with separate conventional electric stimulation devices (external device 10100) respectively, wherein, referring to FIG. 14, the electrode unit 12000 and the control unit 13000 may be modulated to configure a plurality of electrodes 12000a and a plurality of control units 13000a separately.

Herein, it is preferable that the insulation unit 16000 is also modulated for performing insulation, wherein a plurality of insulation portions 16000a are modulated to match with the respective configuration elements.

Particularly, it is preferable that the control unit 13000 in FIG. 14 interlocks with the respective control portions 13000a to be configured to include a function controlling the control portions 13000a integrally.

That is, the plurality of electrode portions 12000a are configured separately by the modulated electric stimulation devices 10100a but may perform a similar specification of the electrode unit 12000. The control portion 13000a performing a function controlling the first electrode 12100 of the electrode portion 12000a to be selectively activated (ON), this function included in the control unit 13000 of the present invention.

Further, the power supply portion may be also configured on the basis of the same technical context as the above.

Referring to FIG. 15, a method for controlling the electric current stimulation device 10000 capable of selectively stimulating a region, configured as the above may comprise steps of measuring a brain wave in real time S10000, determining a stimulation area S20000, determining a selection option S30000 and focusing microcurrent S40000.

In the step of measuring a brain wave in real time S10000, the brain wave measurement unit 14000 measures an arbitrary brain wave from the user in real time.

In the step of determining a stimulation area S20000, the control unit 13000 determines at least one stimulation area A required to be stimulated according to the user's disease in the whole area of the brain based on the brain wave measured in the step of measuring a brain wave in real time S10000.

In the step of determining a selection option S30000, in order to treat the disease by focusing the microcurrents on the stimulation area A, the control unit 13000 determines at least one of two or more of the first electrodes 12100 to be activated (ON) in the plurality of electrodes of the electric unit 120000, the intensity of the microcurrent which the respective electrodes 12100a, 12100b, 12100c, 12100d, 12100e, 12100f of the first electrode 12100 should induce and the route thereof.

In the step of focusing the microcurrents S40000, the electrode unit 12000 induces the microcurrent according to the result determined in the step of determining a selection option S30000 to focus the microcurrent on the stimulation area determined in the step of determining a stimulation area S20000, followed by transferring the microcurrent to the user's brain by the CES method.

Meanwhile, the method for controlling the electric current stimulation device 10000 may further include a step of correcting a selection option.

More particularly, in the step of correcting a selection option S50000, after focusing the microcurrents on the first stimulation area preferentially determined in the stimulation area A, after focusing the microcurrents on the first stimulation area preferentially determined in the aforementioned stimulation area A, the control unit 13000 determine a necessity to change the first stimulation area while continuously monitoring a state of the user's brain on the basis of the brain wave measured in the step of measuring a brain wave in real time.

Further, in the step of correcting a selection option S50000, when it is determined to change the first stimulation area to a new second stimulation area according to the determination result, in order to focus useful microcurrent on the new second stimulation area, changed is at least one of the first electrode 12100, the intensity of the microcurrent which the respective electrodes 12100a, 12100b, 12100c, 12100d, 12100e, 12100f of the first electrode 12100 should induce and the route thereof.

Furthermore, the method for controlling the electric current stimulation device 10000 may further include a step of operating an alarm unit S40100, S50100.

In the step of operating an alarm unit S40100, S50100, the control unit 13000 outputs different alarm signals at each timing for focusing the microcurrents on the stimulation area A in the aforementioned step of focusing the microcurrents S40000 and for changing at least one of the first electrode 12100, the intensity of the microcurrent which the respective electrodes 12100a, 12100b, 12100c, 12100d, 12100e, 12100f of the first electrode 12100 should induce and the route thereof in the step of correcting the selection option, respectively.

Further, in the step of operating an alarm unit S40100, S50100 the alarm unit 15000 releases a preset pattern of sound and light according to the alarm signal output from the control unit 13000.

Accordingly, according to the present invention, the electric current stimulation device 10000 capable of selectively stimulating a region and the control method thereof determine at least one stimulation area A required to be stimulated according to the user's disease and the control unit 13000 determines at least one of two or more of the first electrodes 12100 required to be activated (ON) in the plurality of electrodes, the intensity of the microcurrent which the respective electrodes 12100b, 12100c, 12100d, 12100e, 12100f of the first electrode 12100 should induce and the route thereof, thereby intensively treating a more accurate area in an appropriate level, consequently providing a more efficient treatment environment to the stimulation area.

Further, according to the present invention, the electrode unit 12000 is composed of a plurality of electrodes arranged into a circular form at regular intervals and generates the route for inducing microcurrents which are multi-crossable mutually as the respective electrodes 12100a, 12100b, 12100c, 12100d, 12100e, 12100f are matched with each other to form a plurality of pairs, thereby securing focusing points for the microcurrent and intensity control ranges variously. Accordingly, it is capable of facilitating control of the stimulation intensity more simultaneously with responding to a wider range of the stimulation area.

In addition, according to the present invention, the configuration including a the main body 11000 which is wearable on the head, the brain wave measurement unit 14000 which measures brain wave in real time and the control unit 13000 which controls an electrode unit adaptively according to the measured brain wave may induce AI-based CES therapy, consequently achieving more effective treatment on at least one disease of sleep disorder, depression, dementia, Mild Cognitive Impairment (MCI), anxiety disorder, development disorder, Attention-Deficit/Hyperactivity Disorder (ADHD).

Further, according to the present invention, the insulation unit 16000 insulating the respective electrical configurations with a circuit prevents mutual electrical interference consequently providing a more reliable treatment environment.

Furthermore, according to the present invention, the control unit 13000 outputting the different alarm signals and the alarm unit 15000 releasing the preset pattern of sound and light allow the user to easily recognize treatment in progress and changes in the brain wave audiovisually.

As mentioned above, the detailed description for the disclosed preferable embodiments of the present invention was provided in order to be easily implemented by those skilled in the art. In the above, the preferable embodiments of the present invention were explained with reference to the accompanying drawings, it will be apparent for those skilled in the art that various changes and modification are allowable within the scope of the present invention. For example, those skilled in the art are able to use the respective configurations described in the aforementioned embodiments in a way of combining the same with each other. Thus, the present invention is not limited to the embodiments shown in this application, but granting the widest scope coinciding with principals and novel features disclosed herein.

The present invention may be rectified to different specific forms within the scope of the spirit and essential features. Thus, the above detailed description should not be understood limitedly in all aspects but should be considered as examples. The scope of the present invention should be determined by interpreting accompanying claims rationally, and includes all modifications within the equivalent scope of the present invention. The present invention is not limited to the embodiments shown in this application, but granting the widest scope coinciding with principals and novel features disclosed herein. Further, the present invention may configure embodiments by combining claims which are not in explicit citation relationship in the patent scope or may include new claims through amendments following filing this application.

That which is claimed is:

1. An electric current stimulation device capable of selectively stimulating a region comprising:
a main body;
an electrode unit which is equipped to the main body to contact at least a part of a user's body; and
a control unit which controls the electrode unit to transfer a microcurrent of 1 mA or less by a Cranial Electrotherapy Stimulation (CES) method, wherein
the control unit determines at least one stimulation area required to be stimulated in a whole area of the user's brain according to a type of the user's diseases, and
determines at least one of two or more of first electrodes required to be activated (ON) in a plurality of electrodes, an intensity of the microcurrent which respective electrodes of the first electrode should induce and a route thereof in order to treat the disease by focusing the microcurrents on the stimulation area.

2. The electric current stimulation device capable of selectively stimulating a region according to claim 1 further comprises a brain wave measurement unit which is equipped to the main body to contact with the part of the user's body and measures a brain wave of the user (EEG, Electro Encephalo Graphy), wherein
after focusing the microcurrents on a first stimulation area preferentially determined, the control unit determine a necessity to change the first stimulation area while continuously monitoring a state of the user's brain based on the brain wave measured in the brain wave measurement unit,
when it is determined to change the first stimulation area to a new second stimulation area according to a determination result,
in order to focus the microcurrent on the new second stimulation area, the control unit changes at least one of the first electrode, the intensity of the microcurrent which the respective electrodes of the first electrode should induce and the route thereof.

3. The electric current stimulation device capable of selectively stimulating a region according to claim 2, wherein
at least one disease of sleep disorder, depression, dementia, Mild Cognitive Impairment (MCI), anxiety disorder, development disorder, Attention-Deficit/Hyperactivity Disorder (ADHD) is treated by the Cranial Electrotherapy Stimulation (CES) method.

4. The electric current stimulation device capable of selectively stimulating a region according to claim 3, wherein
whole of the electrode unit is composed of at least eight or more electrodes arranged into a circular form at a regular interval and
the respective electrodes of the first electrode according to the arrangement induces a multi-crossable microcurrent by controlling of the control unit.

5. The electric current stimulation device capable of selectively stimulating a region according to claim 4, wherein
the control unit activates the respective electrode of the first electrode simultaneously (ON).

6. The electric current stimulation device capable of selectively stimulating a region according to claim 4, wherein
the main body is formed into a three-dimensional hemisphere shape having a circumference and is wearable on a head of the user's body and
whole of the electrodes of the electrode unit is arranged along the circumference of the main body.

7. The electric current stimulation device capable of selectively stimulating a region according to claim 1, wherein
a wave form of the microcurrent is any one of DC, AC, random waveform, coded waveform and waveform generated in an external device through wired/wireless connection.

8. The electric current stimulation device capable of selectively stimulating a region according to claim 1, wherein
a pattern of the microcurrent is any one of DC, sine, pulse, chirp, random patterns and a pattern generated in an external device through wired/wireless connection.

9. The electric current stimulation device capable of selectively stimulating a region according to claim 1, wherein
the control unit,
in a case that the microcurrent is a constant current,
calculates an intensity of the microcurrent in a proportion of 100% when passing a center with respect to the stimulation area, and
calculates an intensity of the microcurrent in a proportion of 20% when passing outmost with reference to the stimulation area.

10. The electric current stimulation device capable of selectively stimulating a region according to claim 9, wherein
the control unit controls the intensity of the microcurrent while controlling a voltage taking account into impedance between the respective electrodes of the first electrode.

11. The electric current stimulation device capable of selectively stimulating a region according to claim 2, wherein
the control unit outputs different alarm signals for each case of focusing the microcurrents on the stimulation area and of changing at least one of the first electrode, the intensity of the microcurrent which the respective electrodes should induce and the route thereof, respectively, and
the electric current stimulation device releases a preset pattern of sound and light according to the alarm signal output from the control unit.

12. The electric current stimulation device capable of selectively stimulating a region according to claim 2, wherein
the alarm unit comprises:
a sound output portion which releases a preset pattern of sound according to the alarm signal and acoustically informs the user of treatment in progress and changes in the brain wave; and
a light emitting portion which releases a preset pattern of light according to the alarm signal and visually informs the user of treatment in progress and changes in the brain wave.

13. The electric current stimulation device capable of selectively stimulating a region according to claim 1 further comprises an insulation unit which insulates the electrode unit and the control unit with a circuit to prevent electrical interference.

14. The electric current stimulation device capable of selectively stimulating a region according to claim 13, wherein
the insulation unit comprises:
a first insulation portion which prevents electrical interference for a power signal; and
a second insulation unit which prevents electrical interference for a control signal.

15. The electric current stimulation device capable of selectively stimulating a region according to claim 14, wherein
the first insulation portion is any one of a transformer or a DC-DC converter, and
the second insulation portion is any one of a photo coupler or a signal transformer.
